(19) United States
(12) Patent Application Publication
(10) Pub. No.: US 2007/0099864 A1
(43) Pub. Date: May 3, 2007

(54) Use of o-atp for the treatment of diseases involving angiogenesis

(76) Inventor: Maria Elena Ferrero, Milano (IT)

Correspondence Address: YOUNG & THOMPSON, 745 SOUTH 23RD STREET, 2ND FLOOR, ARLINGTON, VA, 22202, US

(21) Appl. No.: 10/589,621

(22) PCT Filed: Feb. 14, 2005
(86) PCT No.: PCT/EP05/01458

§ 371 Date: Oct. 6, 2006

(30) Foreign Application Priority Data
Feb. 17, 2004 (IT) MI2004A000255

(51) Int. Cl. A61K 38/19; A61K 38/17; A61K 31/7076; A61K 31/401; A61K 31/366; A61K 31/22

(52) U.S. Cl. 514/47; 514/12; 424/85.1; 514/423; 514/460; 514/548

(57) ABSTRACT

The use of o-ATP as a pharmacological agent useful for the treatment of diseases in whose onset or progression angiogenesis is involved, such as ocular diseases, atherosclerotic processes or tumors.

The present invention relates in general to substances that act on angiogenesis. More precisely, the invention relates to the use of o-ATP for the treatment of pathologies that require inhibition of angiogenesis.

### BACKGROUND OF THE INVENTION

Angiogenesis

Proliferation of endothelial cells is responsible for the process of formation of new blood vessels, known as angiogenesis. The newly formed vessels provide nutrients and oxygen to the cells of the tissue wherein angiogenesis occurs. The angiogenetic process is useful, for example, for wound repair, since regenerating tissues necessitate a proper blood supply. On the contrary, angiogenesis is detrimental in the case of tumour diseases, because blood supply facilitates the proliferation of tumour cells. In addition, neoangiogenesis is detrimental when develops into the atherosclerotic plaques; in fact in these structures the generation of new vessels due to VEGF (vascular endothelial growth factor) production by endothelial and other cells, as monocytes/macrophages, supports the preservation of the same plaques. Therefore, the inhibition of endothelial cells proliferation, or anti-angiogenic activity, is of remarkable interest in antitumour and antiatherosclerotic therapies.

o-ATP's Biological Activity

The oxidized form of ATP, known as o-ATP, is characterised by the presence of two aldheyde groups at the positions 2′ and 3′ of the ribofuranosyl ring. It can be prepared by treatment of ATP with a periodic acid salt, as disclosed by P. N. Lowe et al., “Preparation and chemical properties of periodate-oxidized adenosine triphosphate and some related compounds”, Biochemical Society Transactions, vol. 7:1131-1133, 1979.

o-ATP is commonly used as an affinity marker for nucleotide enzymatic sites (Easterbrook-Smith, B., Wallace, J. C. & Keech, D. B. (1976) Eur. J. Biochem. 62, 125-130), thanks to its ability to react with non-protonated lysine residues that are present in the nucleotide sites, forming Schiff's bases or dihydromorpholine-derivatives (Colman, R. F. (1990) in The Enzymes-Sigman, D. S., and Boyer, P. D., eds-Vol 19, pp. 283-323, Academic Press, San Diego). It has also been used to study platelet activation and inhibit ATP-induced stimulation of chicken muscle (Pearce, P. H., Wright, J. M. Egan. C. M. & Scrutton, M. C. (1978) Eur. J. Biochem. 88, 543-554; Thomas, S. A., Zawisa, M. J., Lin, X. & Hume, R. I. (1991) Br. J. Pharmacol. 103, 1963-1969). Furthermore, in macrophage cell lines, o-ATP proved able to block ATP-induced permeabilization of the plasma membrane, reduce the hydrolysis level of exogenous ATP by membrane ecto-ATPases, and inhibit ATP-induced cell swelling, vacuolization and lysis (Murgia et al. The Journal of Biological Chemistry, (1993) by The American Society for Biochemistry and Molecular Biology, inc., Vol. 268, No. 11, pp 8199). It has been suggested that o-ATP has an antagonist activity on the purinergic receptor P2z/P2×7, due to the fact that IL-1β (interleukin 1β) release (which is dependent on LPS=lipopolysaccharide) from microglia cells expressing P22/P2X7 is selectively inhibited by o-ATP (Ferrari D. et al., J. Exp. Med., (1997) Vol. 185, N. 3, Pag. 579-582).

### PRIOR ART

WO 02/11737, in the name of the Applicant, discloses o-ATP antinflammatory and analgesic effect, using unilateral inflammation of rat paw caused by intraplantar injection of complete Freund's adjuvant (CFA) as the experimental model.

### DESCRIPTION OF THE INVENTION

In vitro assays on human umbilical vein endothelial cells (HUVEC), have shown that o-ATP induces a significant reduction of their proliferative capacity, even in the presence of a mitogen. The effect of o-ATP resulted higher than that induced by vasostatin, a known anti-angiogenic compound.

It is therefore object of the present invention the use of o-ATP for the inhibition of angiogenesis. In particular, the invention provides a medicament containing o-ATP as the active principle, useful for the treatment of pathologies, the onset or progression of which involves angiogenesis. The angiogenesis-mediated diseases that can benefit from the treatment with o-ATP according to the invention include neovascularization-induced ocular diseases, such as diabetic retinopathy, macular degeneration, proliferative vitreoretinopathy, glaucoma, atherosclerotic processes and tumours, such as carcinomas, lymphomas, leukaemia, sarcomas, melanomas, gliomas, neuroblastomas and other solid tumours.

For therapeutical use, o-ATP can be formulated with pharmaceutically acceptable carriers and excipients, and administered through the oral, topical or parenteral route. Pharmaceutical forms suitable for the different administration routes comprise tablets, pills, capsules, granulates, powders, suppositories, syrups, solutions, suspensions, creams, ointments, gels, pastes, lotions, emulsions, sprays. Pharmaceutical compositions can be prepared as described in Remington's Pharmaceutical Sciences Handbook, Mack Pub. Co., NY, USA, XVII Ed. The amount of active substance per dose unit ranges from 0.01 to 100 mg per Kg of body weight, to be administered once a day or more according to the type and severity of the pathology. In general the daily dose will range from 1 to 300 mg, preferably from 10 to 100 mg.

In another embodiment, the invention refers to combined preparations of o-ATP and other biologically active substances for the treatment of angiogenesis-mediated pathologies. According to a preferred embodiment, o-ATP is used in combination with antitumour substances such as alkaloids, antibiotics, cytotoxic or cytostatic compounds, antimetabolites, antihormonal agents, alkylating agents, peptides, biological response modulators, cytokines. Alternatively, oATP is used in combination with antiatherosclerotic substances, preferably with lipid lowering drugs or statins.

The different active substances can be administered either simultaneously or separately. The choice of the specific combination of active substances, their dosage and way of administration depend on the specific disease, its resistance to pharmacological treatments, patient's tolerance and other variables to be determined on a case by case basis.

### EXAMPLE 1

### Proliferation Assay

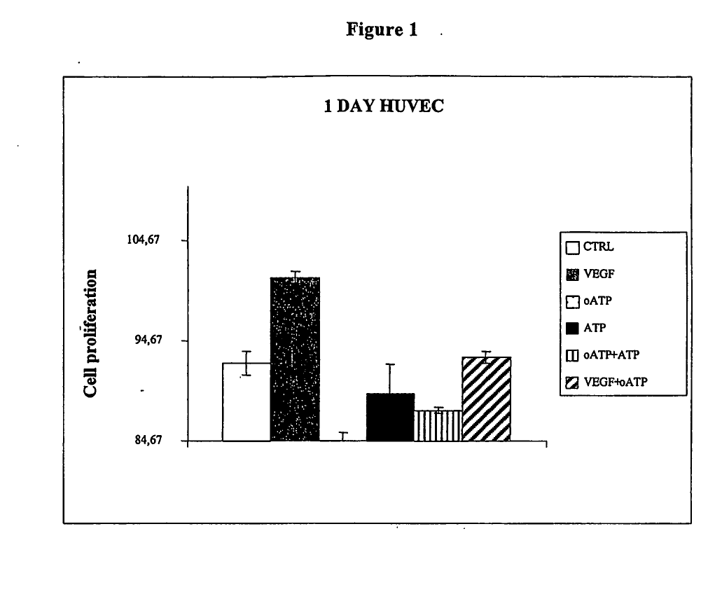

Human endothelial Cells (HUVEC) were isolated from umbilical vein, counted and seeded in a costant number in a 96-wells plates. The cells were cultured as described (Jaffe, E. A. (1984) Biology of Endothelial Cells, Martinus Nighoff Publisher, Boston, USA, pp. 1-260), with or without (control) VEGF (50 ng/ml), in the presence of o-ATP (100 μM), and o-ATP+VEGP. After 24 hours cultivation with or without stimulus, the cells were washed and counted with an optical microscope using a Burker chamber. The results are reported in FIG. 1 and represent the mean±SD of 10 experiments.

### EXAMPLE 2

### Permeability Assay

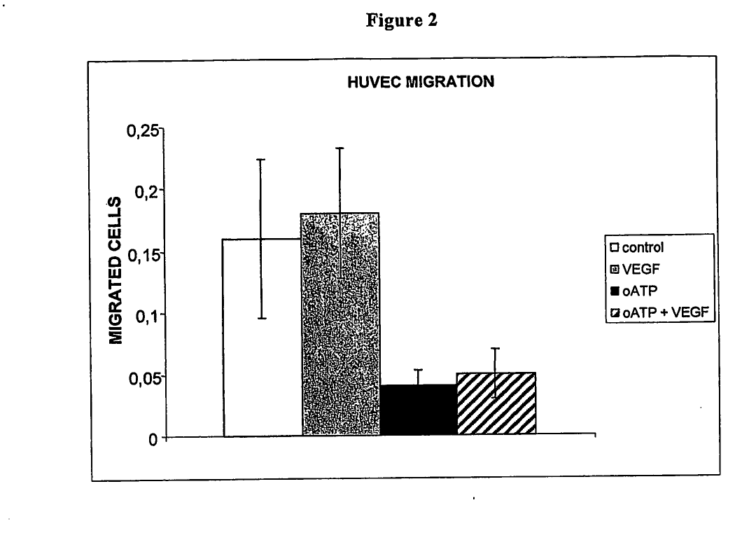

Transwell chambers for cell cultures (polycarbonate filters 0.4 μm, Costar) were used. In short, confluent endothelial cells, in monolayer, were exposed to VEGF, o-ATP, ATP (300 μM), ATP+o-ATP, o-ATP+VEGF (at the previously indicated concentrations) for 1 hr and thoroughly washed. Albumin marked with ¹²⁵I (NEN, Boston, Mass.) was added to the upper compartment; cold albumin (1.5 mg/ml) was added to the culture medium to minimize transcytosis. One hour after the addition of ¹²⁵I-labelled albumin to each well, samples were taken from the lower compartment. The radioactivity of the samples was measured with a gamma counter (Packard, Sterling, Va.). The results, reported in FIG. 2, represent the mean±SD of 10 independent experiments and are expressed as percentage of migrated endothelial cells.

1. A method for administering a composition with antiangiogenic activity, comprising, administering to a subject in need thereof an effective amount of o-ATP.

2. The method according to claim 1, for the treatment of diseases which in their onset or progression involve angiogenesis.

3. The method according to claim 1, for the treatment of ocular diseases, atherosclerotic processes or tumours.

4. The method according to claim 3, for the treatment of carcinomas, lymphomas, leukemias, sarcomas, melanomas, gliomas, neuroblastomas.

5. Therapeutic preparation containing o-ATP in combination with an antitumor substance selected from cytotoxic or cytostatic compounds, antimetabolites, alkaloids, antibiotics, alkylating agents, peptides, biological response modulators and cytokines, for simultaneous, separate or sequential use in tumour treatment.

6. Therapeutic preparation containing o-ATP in combination with an antiatherosclerotic substance selected from lipid lowering drugs and statins, for simultaneous, separate or sequential use in the treatment of atherosclerotic processes.
